(19) United States
(12) Patent Application Publication
(10) Pub. No.: US 2006/0223183 A1
(43) Pub. Date: Oct. 5, 2006

(54) Gamma irradiation of frozen feeder cells

(76) Inventor: Steven E. Bates, Duarte, CA (US)

Correspondence Address: PERKINS COIE LLP, POST OFFICE BOX 1208, SEATTLE, WA, 98111-1208, US

(21) Appl. No.: 11/372,912

(22) Filed: Mar. 10, 2006

Related U.S. Application Data
(60) Provisional application No. 60/660,709, filed on Mar. 11, 2005.
(60) Provisional application No. 60/664,569, filed on Mar. 22, 2005.

(51) Int. Cl. C12N 15/01; C12N 5/06; C12N 5/08

(52) U.S. Cl. 435/446; 435/354; 435/366

(57) ABSTRACT

Gamma irradiation of frozen feeder cells improves their function, uniformity and extends their usable lifespan, thereby allowing a more robust and flexible stem cell culture system. Feeder cells that are irradiated while frozen do not suffer the reduced functional lifespan of feeder cells that are irradiated and then frozen and do not experience the effects of the radiation until they are thawed. The present invention has the significant advantage of improving the preparation of feeder cells, which simplifies the culture of target cells, thus maximizing research efforts and cutting costs.

### CROSS-REFERENCE TO RELATED APPLICATIONS

This application claims the benefit of U.S. Provisional Patent Application No. 60/660,709, filed Mar. 11, 2005 and U.S. Provisional Patent Application No. 60/664,569, filed Mar. 22, 2005. The disclosures of these applications are incorporated herein by reference in their entirety.

### GOVERNMENT INTEREST

NIH Grants RO1-CA090819, RO1-ES06070, and U42 RR16607 were used, in part, in support of the present invention. The government may have rights in the present invention.

### BACKGROUND

Although claims exist of feeder cell-free culture of mammalian embryonic stem cells (1, 2), these techniques require expensive growth factors and extracellular matrices, which are often derived from feeder cells. For the foreseeable future, feeder cell-based embryonic stem cell culture will remain the standard of comparison for all claims of improvement in the art. Feeder cells are often utilized to support the growth of target cells such as embryonic stem cells, embryonal carcinoma or teratoma, and transfected cells and are especially useful when attempting to clone target cells at low or single cell density. Enzymatically dissociated mouse embryos are typically used as a source of feeder cells, and are termed Mouse Embryonic Fibroblasts (MEF). However, other normal cell types can also be used.

In order to prevent overgrowth of target cells by the feeder cells, the feeder cells must be rendered mitotically incompetent. This is achieved by treatment of the feeder cells with either Mitomycin C or ionizing radiation (usually gamma radiation), after which the cells are used as feeders or are frozen for later use. Gamma irradiation is preferable to Mitomycin-C treatment of feeder cells because of residual drug toxicity to the target cells. Because gamma sources are relatively expensive and inaccessible, the Mitomycin-C method is frequently used, although it is the inferior alternative. If ionizing radiation is used, cultured cells must be taken from incubators, transported to a gamma source, irradiated, returned to culture, and later harvested and/or frozen. During this process, such factors as changes in temperature, pH, shaking, drying, or light exposure are deleterious conditions for the feeder cells, and shorten their useful lifespan. This method also increases the risk of contamination during transport and usually necessitates the use of flasks during irradiation.

Even the highest quality feeder cells frequently cease to function before the target cell or colony is ready to be passed. Further, some protocols require fresh feeder cells two or three times per week. Fresh feeder cells are not inoculated into a culture of exhausted feeder cells, because the latter are in various stages of physical disintegration and promote the differentiation of the target cells. The current art requires that freshly inactivated feeder cells be available at regular intervals during stem cell culture, requiring frequent, easy access to a gamma source and timed pregnancies to produce fresh feeder cells. Thus, the duration of feeder cell function, not the optimal treatment of the target cells, is a limiting factor of stem cell culture by requiring physical passage of the target cells to fresh feeder cultures. Because of this limitation, any innovation which extends feeder cell longevity and function is highly desirable and a needed improvement in the art.

### SUMMARY OF THE INVENTION

Applying gamma radiation to frozen feeder cells renders them growth-limited and better suited for use in supporting target cells. A significant and unexpected advantage of the present invention is that the feeder cells that are irradiated while frozen do not suffer the reduced functional lifespan of feeder cells that are irradiated and then frozen. Importantly, the feeder cells do not experience the effects of the radiation until they are thawed.

### BRIEF DESCRIPTION OF DRAWINGS

FIG. 1 is an image of Mouse Embryonic Fibroblast Cells (MEF) treated with 30 Gray of gamma radiation while frozen. Frozen-irradiated cells are visually indistinguishable from cultured-irradiated cells after thawing, but possess a longer usable lifespan. This image shows an anaphase frozen-irradiated MEF cell amid the debris from the prior twelve-hour thaw. Although many cells that survive isolation from the embryo are killed by freezing, those that do survive are the youngest possible, and the most serviceable for feeder applications. Higher doses of gamma radiation can prevent any cell division after thawing. However, the lowest gamma dose which limits, but does not eliminate growth, yields the longest feeder lifespan.

FIG. 2 is an image of Human Telomerase Transformed Fibroblast Cells treated with 55 Gray of gamma radiation while frozen. Minimally, but sufficiently, irradiated cells are capable of cell division, but at a greatly reduced rate, which prevents overgrowth of the target cells by the feeder cells. Established cell lines such as htertG can have 85% survival from freezing and can be observed to divide at 8 hours post-thaw.

FIG. 3 is an image of Mouse Embryonic Stem Cell (ESC) line W-95 co-cultured with frozen-irradiated MEF cells (mouse stem/mouse feeder). MEF feeder cells are frozen, irradiated with 30 Gray of gamma radiation, and thawed. The Mouse ES cells are plated and the co-culture is then photographed. In the image center is an ESC colony of approximately 70 cells. The cells are small, regularly shaped, and the colony has a round, defined border, which are the morphological characteristics indicative of undifferentiated embryonic stem cells. Peripheral to the ESC colony are MEF feeder cells derived from CF-1 mice, which were frozen 48 hours after collagenase digestion of embryos.

FIG. 4 is an image of Mouse Embryonic Stem Cell line W-95 co-cultured with frozen-irradiated htertG feeder cells (mouse stem/human feeder). htertG feeder cells are frozen, irradiated with 55 Gray of gamma radiation, and thawed. Mouse ES cells are then plated and the co-culture is photographed.

FIG. 5 is an image of Human Embryonic Stem Cell line HSF-6 co-cultured with frozen irradiated MEF feeder cells (human stem/mouse feeder). MEF feeder cells are frozen, irradiated with 30 Grey of gamma radiation, and thawed. Human ES cells are thawed and the co-culture is photographed.

FIG. 6 is an image of Human Embryonic Stem Cell line HSF-6 co-cultured with frozen irradiated HF-55 feeder cells (human stem/human feeder). HF-55 feeder cells are frozen, irradiated with 50 Gray of gamma radiation, and thawed. Human ES cells are thawed and the co-culture is photographed.

Definitions

The present invention may be better understood in light of the following definitions:

A “clone” is a proliferating lineage of cells which retain desired characteristics, and are derived from a single cell, or a small number of founding cells.

“Confluence” is the proportion of cell culture surface area occupied by attached, spread cells. 100% confluence of MEF cells is approximately 120,000 cells per square centimeter.

“Contamination” is the colonization of the culture system by undesired organisms such as mycoplasma, bacteria, yeast, fungi, or other mammalian cells. Feeder cell inactivation is required to prevent overgrowth or contamination of embryonic stem cell cultures by feeder cells.

“Culture passage” occurs when cells grow to a certain density and must be transferred to new cultures at lower densities. Passage or transfer of cell cultures is required before the feeder cells fail to function, and may be effected by scraping or enzymatic detachment of cells.

The “culture substrate” is the lower, interior surface of the culture vessel, which is typically glass or polystyrene. The culture substrate may be further treated or coated to promote or prevent cell attachment.

“Cultured cells” are typically mammalian cells attached to culture substrates and maintained at 37° C. in conventional cell culture medium such as DMEM, F-12, RPMI 1640, or MCDB 153.

“Cultured-irradiated cells” are cells that have been exposed to a dose of gamma radiation while attached to flasks or dishes rendering the cells mitotically incompetent. In this case, gamma damage to the cells begins immediately and cannot be delayed.

“Differentiation” is the commitment of a lineage or clone of cells to become a specific cell or tissue type. Differentiation is synonymous with a loss of stem cell characteristics.

“Feeder cell lifespan” is the interval between irradiation and the cessation of feeder cell function. By delaying the onset of this interval until the time of thawing, the frozen-irradiated technique allows the maximum possible feeder cell lifespan.

“Frozen cells” are cultured cells that have been harvested, concentrated, resuspended in cryoprotectant medium and dispensed in vials or ampoules. These are frozen and stored until needed.

“Frozen-irradiated cells” are frozen cells that are exposed to a dose of gamma radiation while in the frozen state rendering the cells mitotically incompetent. Frozen cells may be packed in crushed dry ice, delivered, irradiated, returned to liquid nitrogen for storage and later use or distribution.

“Freezing” is a process of cooling and storing cells at very low temperatures to maintain cell viability. The technique of cooling and storing cells at a very low temperature permits high rates of cell survivability upon thawing. One substance commonly used in freezing cells is liquid nitrogen which has a temperature of about negative 196° C.

“Gamma induced damage” in mammalian cells is caused by the passage of high energy, short wavelength photons, and other subatomic particles which scatter electrons from atoms and molecules through which they pass, producing trails of peroxides, radicals, and other chemically reactive, cytotoxic species.

A “gamma source” is a device allowing exposure of experimental materials, cells or organisms to specific doses of gamma radiation. The Shephard Mark 1 source utilizes radiation from Cesium 137.

“Stem cell characteristics” (also known as “stem cell phenotypes”) include such features as cell morphology, mitotic rate, colony shape, the presence or absence of specific surface antigens, and the capacity to support viral replication.

“Target cells” are cells requiring nutritional support of feeder cells in order to prevent differentiation or extinction. Such cell types include embryonic stem cells, cells transfected with selectable constructs, and cells grown under cloning conditions.

### DETAILED DESCRIPTION OF THE INVENTION

A first aspect of the invention exploits the temporal relationship between the exposure of frozen cells to ionizing radiation, and the subsequent thawing of such cells. The present invention is based on the discovery that gamma irradiated frozen cells do not respond to the gamma induced damage until after they are thawed thus producing improved feeder cells.

A second aspect of the present invention utilizes the earliest part of the feeder cell lifespan. Because feeder cell function deteriorates after lethal gamma irradiation, such function is greatest at the time of exposure. The irradiation of frozen cells effectively extends the early part of feeder cell lifespan by several hours or days because they do not deteriorate while they remain frozen. This is a critical concern, since the usable feeder cell lifespan is only 2-5 days after irradiation. Thus, a natural definitive starting point for all analysis of feeder cell function is possible by means of this technology. Real comparisons of the feeder function of diverse cell types (from different tissues, organs, species, or combinations of such cell types) are now possible. Theoretically, any cell type having functional cell cycle checkpoint genes could serve as a feeder cell.

A third aspect of the present invention provides a method of research on the early biochemical responses of cultured cells to gamma radiation, based on an absolute starting point in time, since frozen-irradiated cells do not begin to deteriorate until they are thawed. The earliest responding parts of DNA damage recognition systems and commitment to either repair or apoptosis pathways after gamma damage may now be studied more easily.

A fourth aspect of the invention involves the reestablishment of cellular adhesion and gamma damage recognition. An adherent cell type is unattached to a substrate when frozen. Once thawed, the processes of attachment and gamma damage recognition in frozen-irradiated cells commence simultaneously. All five lethally irradiated mammalian cell types studied were found to be unaffected with respect to their ability to attach and spread on the culture substrate. Attachment and spreading are cytoskeletal functions involving the reorganization of actins, microtubules and many other proteins which mediate intracellular communication and vesicle trafficking. The method of present invention distinguishes cytoplasmic damage perception from that of the nucleus, if such perception is related to attachment.

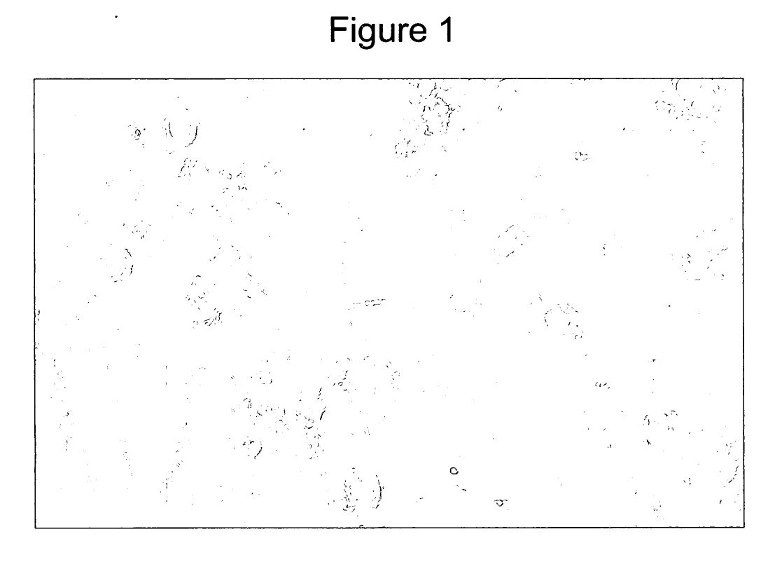

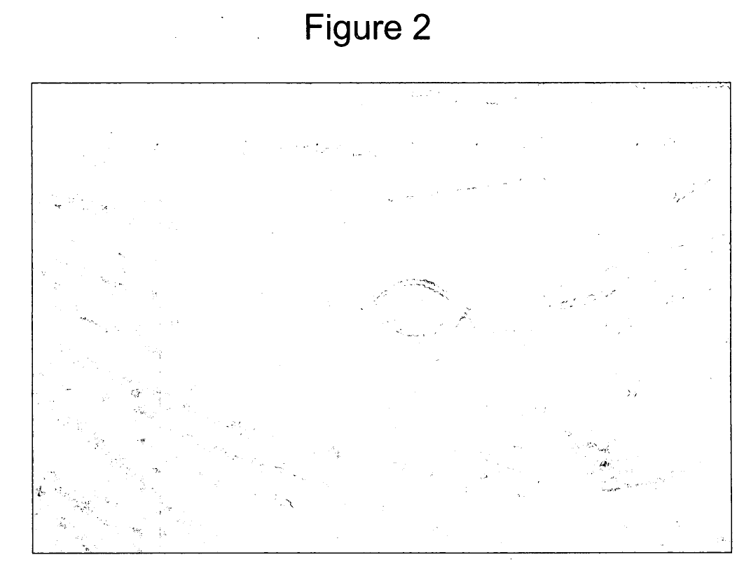

The present invention teaches a method of applying gamma radiation to frozen feeder cells to ensure that they are rendered growth-limited. The freezing process reduces the recovery of viable cells by at least 10%. Young, healthy, actively dividing cells are preferred for freezing as they can recover from the stress of freezing with only a small reduction in growth rate. Injured, insulted or senescent cells do not freeze well. Consequently, post-thaw viability of cultured-irradiated cells is approximately 20%-50% lower than that of frozen-irradiated cells. This may be because the freezing of previously inactivated cells adds a further deleterious stress to these cells, which reduces their viability when thawed. Irradiation of cells in the frozen state circumvents this problem. FIGS. 1 and 2 are photographs showing cells that were treated with gamma radiation while frozen.

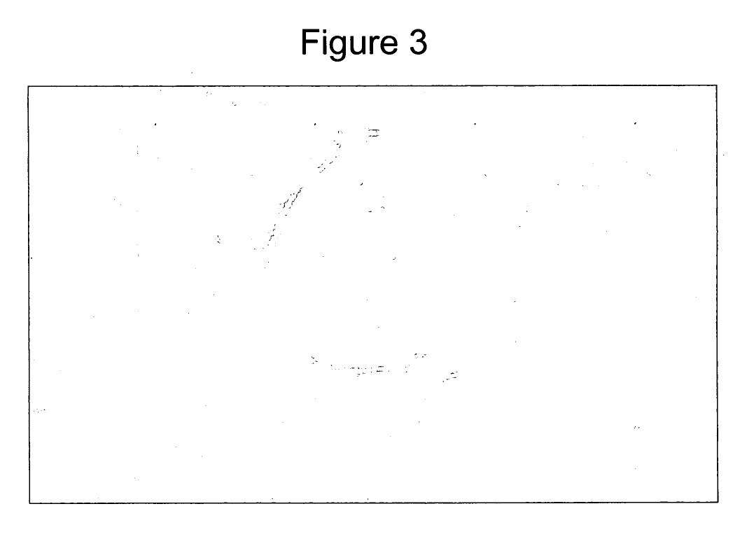

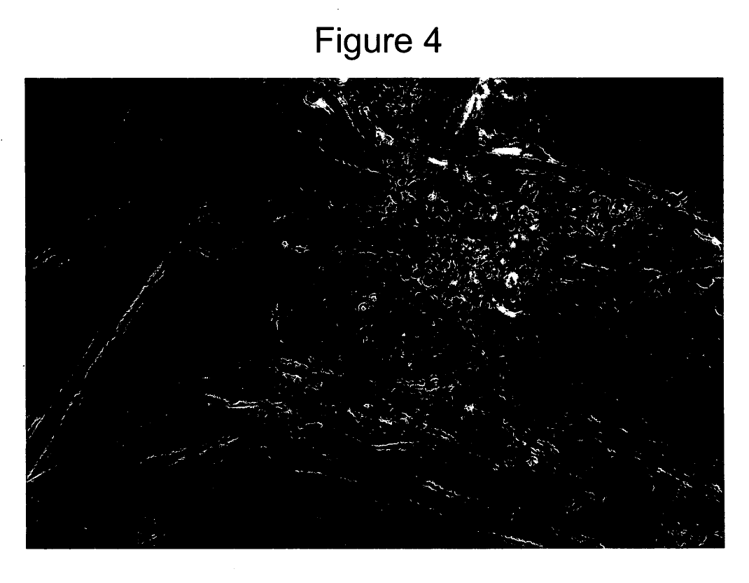

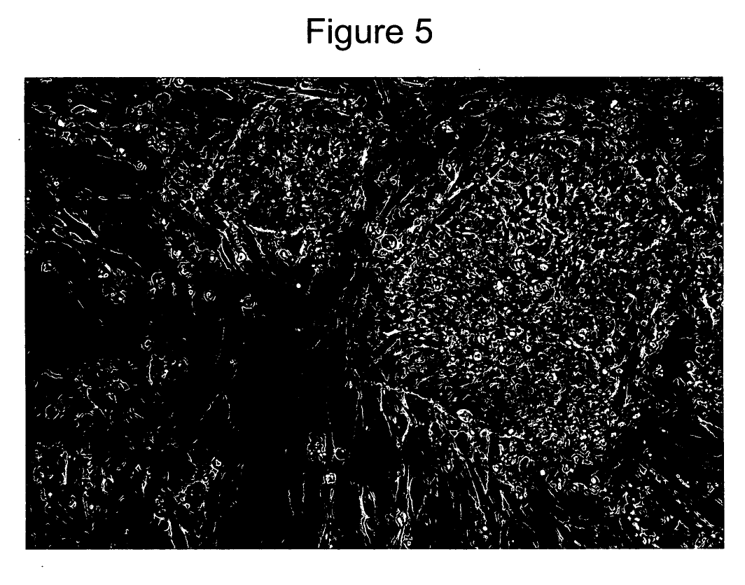

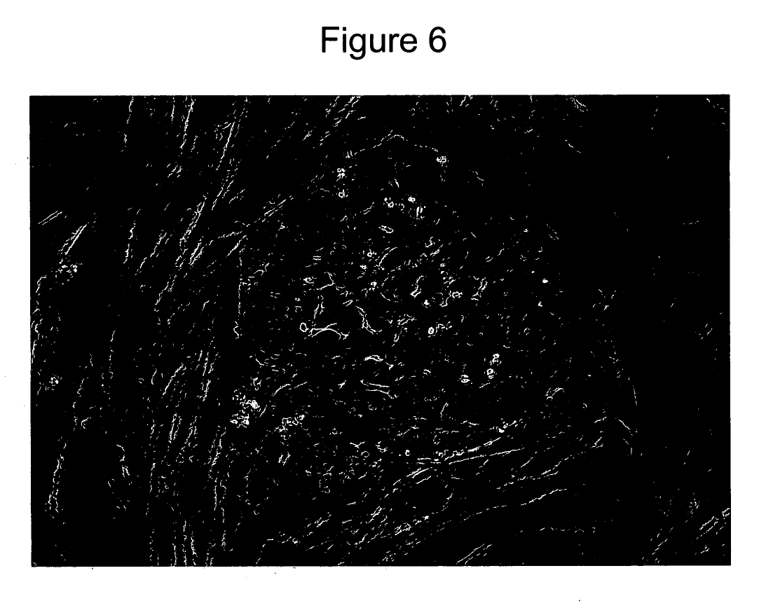

MEF functions decrease with time after isolation from the embryos, culture age, and increasing gamma dosage. This dose must be kept as low as possible in order to obtain the maximum feeder cell lifespan, with a minimum exposure variation between different cells in the population, and yet be sufficiently high to prevent feeder cell proliferation. The current practice of cultured irradiation followed by freezing wastes at least one culture passage, because cells are attached to flasks during irradiation and must be subsequently transferred to the target cell dish. Even if feeder cells are harvested, irradiated in suspension, and then plated (1), the number of cells which can be treated at one time is far less than with the frozen-irradiated technique, and unavoidable deterioration of feeder function commences immediately upon irradiation. The frozen-irradiated technique yields many hours or days in the earliest, most valuable, period of the feeder cell lifespan which is otherwise wasted by traditional feeder cell generating techniques. Once frozen-irradiated cells are thawed and transferred to a culture vessel, they attach and spread on the culture vessel substrate, which typically takes less than four hours. They are ready to function as feeder cells and can receive the target cells. FIGS. 3-6 are photographs showing various target cell lines co-cultured with feeder cells.

A far greater number of cells can be irradiated at one time in the frozen state. One frozen ampoule can contain sufficient numbers of cells to occupy tens to hundreds of square centimeters of cell culture area, depending on the desired final confluence. Because of liquid containment and contamination concerns, most researchers irradiate cultured cells in flasks or detached cells in suspension in tubes. However, for culture-irradiated cells, care must be taken to ensure that each cell receive a minimal radiation dose to render it mitotically incompetent and uniform irradiation of each cell is difficult. The gamma flux variation may be greater than 30% between remote corners of the irradiation chamber. Even if irradiated on a rotating platform to smooth the exposure, attached cells in different parts of a culture vessel experience different dosages of radiation.

Because presumptive feeder cells must be irradiated to an extent to render them mitotically incompetent, and yet allow them to retain their function as feeder cells, the uniformity of radiation dosage becomes a critical issue. Frozen ampoules are much smaller than culture flasks or dishes and many can be easily positioned at the prime focus of the gamma source where the gamma flux variation is less than 3%. This frozen irradiated strategy allows cells to be uniformly exposed to a minimal level of radiation required to render the cells mitotically incompetent.

### EXPERIMENTS

To prepare frozen irradiated ampoules of desired cell type(s), healthy cells were harvested, counted and frozen in liquid nitrogen. One ampoule of each set of frozen ampoules was thawed, the viability was measured by trypan blue exclusion, and the confluence level of the resultant culture was estimated. Freezes from which at least 70% of the cells survive are preferred.

The Shepherd Irradiator has a variety of configurations, and includes a carousel with circular aluminum trays which can be stacked and rotated during exposure. Crushed dry ice is used to fill one tray, and the frozen ampoules are placed in the center so that the bottoms of the ampoules are at the level of, and at a known distance from, the gamma source. The tray of dry ice and ampoules is slowly rotated thus insuring a uniform exposure during the desired exposure. Gamma flux decreases slowly and predictably with time, and is periodically recalculated from a half-life nomograph provided by the manufacturer. Mouse Embryonic Fibroblasts are exposed to around 30 Grey, whereas Human Skin Fibroblasts are exposed to 45-60 Grey.

Because gamma radiation damage is additive in the frozen state, different gamma dosages can be administered by removal of lower dosage ampoules at earlier times during the irradiation. As long as the cells remain frozen, one exposure of 60 Grey is equivalent to four doses of 15 Grey, for example. If different gamma dosages are desired, the ampoules can be colored or positionally coded, and the lower dosage ampoules are removed first. Irradiated ampoules are maintained on crushed dry ice during transport and irradiation, and are returned to liquid nitrogen for storage. The frozen-irradiated feeder cells are now ready for use. Because the cellular response to gamma radiation does not occur until after thawing, a library of feeder cells can be established for use at a later date. This vastly simplifies the provision of “inactive” feeder cells for stem cells culture.

For the materials, Mouse Embryonic Fibroblasts (MEF) derived from CF-1 mice purchased from Charles River Laboratories. Eight to thirteen day p.c. embryos were used to produce MEF cells. MEF cells immortalized by virus (MEFv) were a gift from Dr. L. Samson of M.I.T. HF-55 and HF-57 are normal human foreskin fibroblast cell lines. hTert G is HF-57 transformed by pBABEhygro, a construct which confers overproduction of telomerase, thereby extending the in vitro lifespan of the cells. Source tissue was obtained from Arcadia Methodist Hospital. The HF-57 cell line was derived in 1995, and transformed by telomerase over expression in March 2001 S. E. Bates at City of Hope. The pBABEhygro construct was a gift from Dr. Robert Weinberg of the Massachusetts Institute of Technology.

### REFERENCES CITED

1. U.S. Pat. No. 6,800,480; Bodnar, et al. Methods and Materials for the growth of primate derived primordial stem cells in feeder-free culture.

2. Rosler, E. S., G. J. Fisk, X. Ares, J. Irving, T. Miura, M. S. Rao, and M. K. Carpenter, Long-term culture of human embryonic stem cells in feeder-free conditions. Dev Dyn, 2004. 229(2): p. 259-74.

### OTHER REFERENCES

1. Kusakabe, H. and Y. Kamiguchi, Chromosomal integrity of freeze-dried mouse spermatozoa after 137Cs gamma irradiation. Mutat Res, 2004. 556(1-2): p. 163-168.

2. Ankotu, S. and K. Gholipour-Khalili, Effect of oxygen on bacteria and cultured mammalian cells irradiated in the frozen state. Int J Radiat Biol Relat Stud Phys Chem Med. 1977 August;32(2):145-52.

1. Improved feeder cells prepared by a process comprising the steps of
a. freezing mammalian cells, and
b. subsequently irradiating the mammalian cells,
so as to produce improved feeder cells.

2. A method of producing feeder cells with improved function, comprising first freezing and subsequently irradiating mammalian cells.

3. A method of producing feeder cells with increased longevity, comprising first freezing and subsequently irradiating mammalian cells.

4. A method of producing feeder cells with increased viability, comprising first freezing and subsequently irradiating mammalian cells.

5. The method of claim 1, 2, 3 or 4, wherein the cells are irradiated with gamma radiation.

6. The method of claim 5, wherein the dose of radiation administered to the cells is 30 Gray or is between 15 and 65 Gray, 30 and 35 Gray or 50 and 65 Gray.

7. The method of claim 1, 2, 3 or 4, wherein the mammalian cells are MEF-CF1, MEFv, HF-55, HF-57 or htertG.

8. Improved feeder cells, wherein
a. mouse embryonic fibroblasts are frozen and then exposed to 30 Gray of gamma radiation, or
b. human skin fibroblasts are frozen and then exposed to between 50 and 60 Gray of gamma radiation,
so as to produce improved feeder cells.
